(19) United States
(12) Patent Application Publication
(10) Pub. No.: US 2008/0145857 A1
(43) Pub. Date: Jun. 19, 2008

(54) MICROORGANISM DETECTION SYSTEM

(76) Inventors: Hisao SAITO, Matsudo (JP); Masahiro KURIHARA, Yokohama (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/955,448

(22) Filed: Dec. 13, 2007

(30) Foreign Application Priority Data
Dec. 14, 2006 (JP) 2006-337088

(51) Int. Cl. C12M 1/34; B01J 19/00; C12Q 1/68

(52) U.S. Cl. 435/6; 422/68.1; 435/288.7

(57) ABSTRACT

A system for detecting microorganisms from a solid or liquid sample by using gene analysis is provided.

A microorganism detection system in the present invention has an analysis chip and an analyzer. Necessary reagents are stored in advance in the analysis chip. A sample is injected into the analysis chip, which is then inserted into the analyzer. The analyzer has a pressure source for supplying a gas under pressure to the analysis chip and a low pressure source for discharging a gas from the analysis chip. The analysis chip has a sample reservoir and the sample reservoir has a filter. Crushed substance contained in the sample is removed by the filter. The analysis chip is further provided with valves in an upstream channel and a downstream channel of the sample reservoir. The valves have a polymer filled in the channel. The polymer is dissolved by heating to open the valve.

### BACKGROUND OF THE INVENTION

1. Field of the Invention

The present invention relates to a microorganism detection system for detecting microorganisms in a sample, and in particular, relates to a microorganism detection system for indirectly detecting microorganisms by using gene analysis.

2. Description of the Related Art

In recent years, instead of directly detecting a microorganism, a method of detecting the existence of microorganism by multiplying a gene of the microorganism and analyzing the multiplied gene has been developed as a detection method of microorganisms. Such a detection method of microorganisms has an advantage that, when compared with the method of directly detecting microorganisms, operations and processing are easy and inexpensive. In addition, if gene analysis of a microorganism is a final objective, such a method can perform processing from detection of the microorganism to acquisition of a result of gene analysis automatically and efficiently.

Japanese Patent Application Laid-Open No. 2005-65607 describes a system that detects microorganisms by capturing a microorganism floating in the air, extracting genes of the captured microorganism, and multiplying and analyzing the genes.

The system described in Japanese Patent Application Laid-Open No. 2005-65607 is a system for capturing microorganisms floating in the air and is not intended for detecting microorganisms in a solid or a liquid.

In recent years, it is becoming increasingly necessary to detect microorganisms in a solid or a liquid such as food. However, neither a method nor a system for detecting microorganisms in a solid or a liquid by using gene analysis has been developed.

An object of the present invention is to provide a system for detecting microorganisms from a solid or liquid sample by using gene analysis.

### SUMMARY OF THE INVENTION

A microorganism detection system according to the present invention has an analysis chip and an analyzer. Necessary reagents are stored in advance in the analysis chip. A sample is injected into the analysis chip, which is then inserted into the analyzer. The analyzer has a pressure source for supplying a gas under pressure to the analysis chip and a low pressure source for discharging the gas from the analysis chip.

The analyzer is provided with a photodetector for detecting genes in a reaction tank of the analysis chip.

The analysis chip is provided with a sample reservoir and the sample reservoir is provided with a filter. Crushed substance contained in the sample is removed by the filter. The analysis chip is further provided with valves in an upstream channel and a downstream channel of the sample reservoir. The valves have a polymer filled in the channel. The polymer is dissolved by heating to open the valve.

According to the present invention, a system for detecting microorganisms from a solid or liquid sample by using gene analysis can be provided.

### BRIEF DESCRIPTION OF THE DRAWINGS

FIG. 1 is a diagram showing a configuration of bacteria detection system according to the present invention;

FIG. 2 is a diagram showing a procedure for bacteria detection by the bacteria detection system of the present invention;

FIG. 3 is a diagram showing the configuration of an analysis chip of the present invention;

FIG. 4 is a diagram showing a sectional configuration of a sample reservoir of the analysis chip of the present invention;

FIG. 5 is a diagram for illustrating an operation of the sample reservoir of the analysis chip of the present invention;

FIG. 6 is a diagram showing a weir of a gene extraction area of the analysis chip of the present invention;

FIG. 7 is a diagram showing the configuration of a reagent storage tank of the analysis chip of the present invention;

FIG. 8 is a diagram showing an appearance of an analyzer of the present invention;

FIG. 9 is a diagram showing the configuration of the analyzer of the present invention; and

FIG. 10 is a diagram showing the configuration of a substrate of the analyzer of the present invention.

### DESCRIPTION OF THE PREFERRED EMBODIMENTS

Embodiments of the present invention will be described below. Incidentally, the present invention is not limited to modes disclosed by each embodiment and allows modifications based on publicly known technologies and the like.

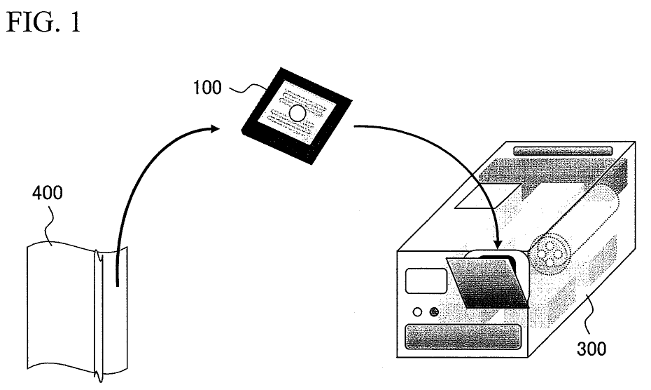

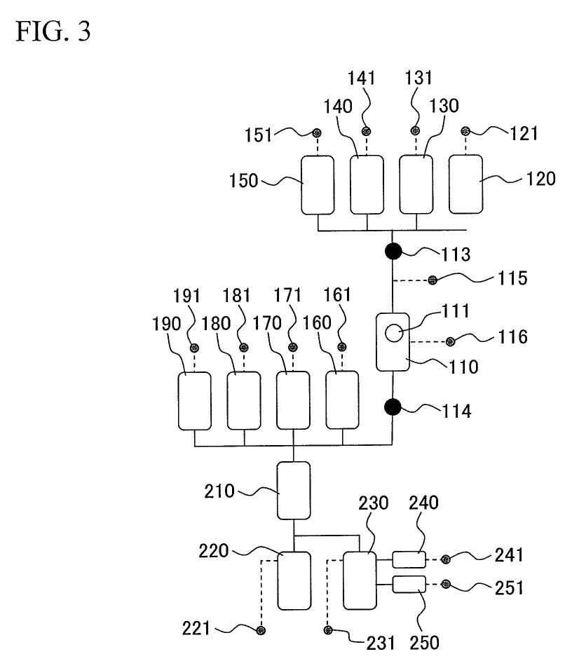

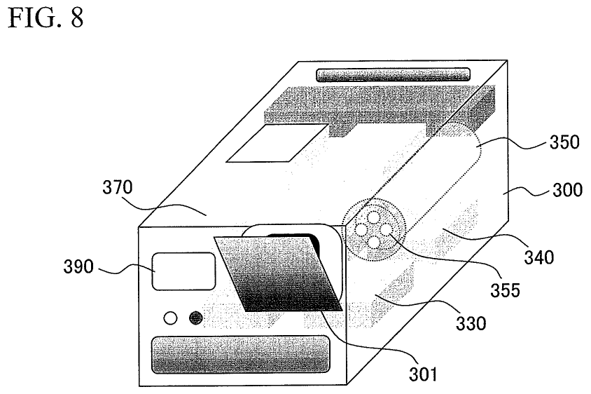

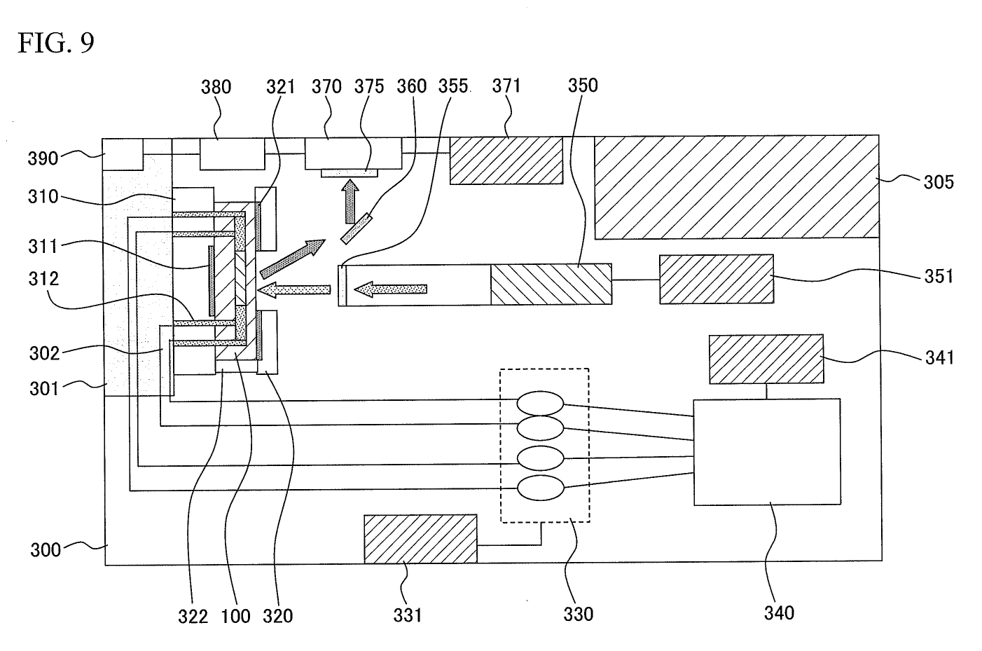

An overview of a microorganism detection system according to the present invention will be provided with reference to FIG. 1. The microorganism detection system in the present embodiment has an analysis chip 100 and an analyzer 300. Details of the analysis chip 100 will be described below with reference to FIG. 3. Details of the analyzer 300 will be described below with reference to FIGS. 8 and 9.

In the detection method of bacteria according to the present invention, genes are extracted by a generally known solid phase extraction method. The solid phase extraction method is an extraction method by which a gene is extracted by causing the gene to specifically bind to a solid surface and causing only the gene, as distinguished from other substances, to elute in an aqueous solution. Further, in the present invention, the gene is amplified by using a polymerase chain reaction.

As illustrated in FIG. 1, an appropriate size of solid sample and distilled water are put into a crush bag 400. An impact of an appropriate force is given to the crush bag 400. The sample is thereby crushed to generate a suspension of the sample. A sample solution is extracted from the crush bag 400 before being injected into the analysis chip 100.

The analysis chip 100 is set to the analyzer 300. Bacteria contained in the sample will be detected by the analyzer 300. When detection of bacteria is finished, the analysis chip 100 is taken out of the analyzer 300 and disposed of.

When a sample is injected into the analysis chip 100, the sample inside the analysis chip 100 will thereafter never be taken out of the analysis chip. Therefore, an operator will never come into contact with the sample during or after analysis. Even if a dangerous bacterium is contained in the sample, the operator will be safe.

The analysis chip 100 contains all necessary reagents required for processes ranging from preprocessing to detection of bacteria. Therefore, a complicated operation of dispensing of reagents can be omitted.

In the present embodiment, the analysis chip 100 is the only waste. The analysis chip 100 is made of material that can be burned up. By disposing of the analysis chip 100 by burning, danger of secondary pollution can be reduced. The analysis chip 100 is disposable. However, the analysis chip 100 contains only reagents for one inspection and therefore, wasteful use of reagents can be prevented. Using the microorganism gene analysis system of the present invention, a high-level bacteria inspection at the gene level can easily be performed anywhere, indoors or outdoors.

The analyzer 300 in the present embodiment holds the analysis chip 100 by keeping the analysis chip 100 standing. That is, the analyzer 300 is a vertical type. However, the analyzer 300 can also be used as a horizontal type in which the analysis chip 100 is held in a horizontal position. If not specifically mentioned, the analyzer 300 in the present embodiment will be described below as a vertical type.

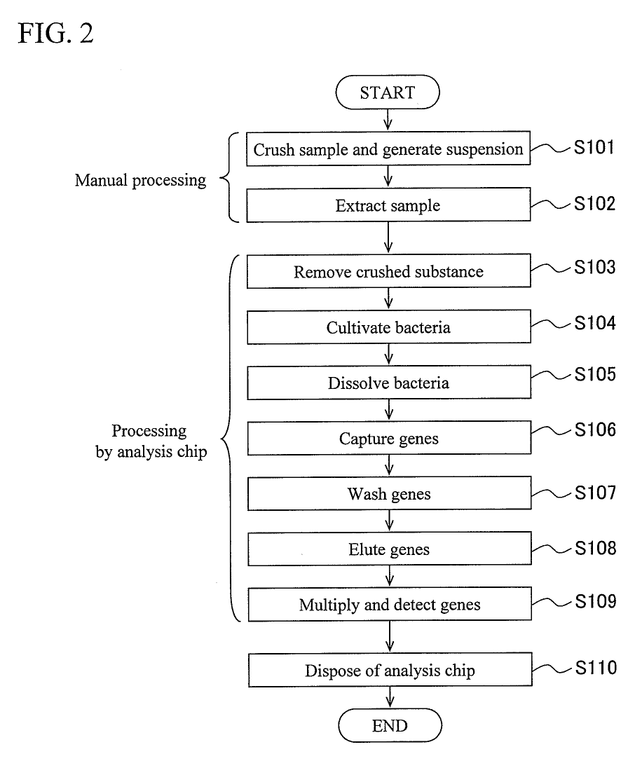

An example of the microorganism detection method according to the present invention will be described with reference to FIG. 2. In step S101, a sample is crushed and a suspension is generated. If the sample is solid, the sample is first cut into cubic pieces measuring several centimeters per side before being put into a crush bag. Distilled water is added to the bag and then the bag is hit manually or using an apparatus for 5 to 10 minutes. The sample in the bag is thereby crushed, producing fragments of 1 mm or less. Bacteria contained on the sample will be suspended in water. A stomaching bag is preferably used because crushed substance measuring 100 μm or larger is filtered by the stomaching bag. The crushing process may be omitted for a liquid sample.

In step S102, the sample is extracted. A predetermined amount of sample is extracted from the bag. In step S103, crushed substance is removed. If crushed substance originating from the solid sample remains, an amplification reaction of bacteria genes is inhibited by proteins in the crushed substance. For this reason, substance (20 μm or more) whose size is equal to or larger than bacteria is removed.

In step S104, bacteria are cultivated. By increasing the number of bacteria through cultivation, bacteria on the order of several bacteria can be detected. If a bacterium is dangerous, only several such bacteria could be fatal. In ordinary cultivation, the number of bacteria is increased until a colony of bacteria is formed in a full day. However, bacteria are here cultivated for a short period of time until the number of bacteria is increased up to about 100. If it is already known that the number of bacteria to be analyzed is sufficiently large, like Escherichia coli in feces, the cultivating process of bacteria may be omitted.

In step S105, bacteria are dissolved. A solution containing chaotropic ions is mixed with the sample. The chaotropic ion is a monovalent anion having a large molecular diameter. The cell membrane of bacteria is destroyed by action of chaotropic ions. Chaotropic ions denature many proteins contained in the sample and have a function to inhibit action of nuclease (enzyme for decomposing nucleic acid).

In step S106, genes are captured. Gene-binding carriers are used for capturing genes. Silica wool, glass wool, glass fiber, a glass bead, a glass filter or the like is used as a gene-binding carrier. The sample containing dissolved bacteria is brought into contact with the gene-binding carriers. By action of the chaotropic ions, a gene is specifically bound to the gene-binding carrier. Generally, the sample is caused to pass through the gene-binding carriers.

In step S107, genes are washed. Proteins, chaotropic ions and the like adhering to the gene-binding carriers and to genes are washed away. High-concentration ethanol is used as a washing solution. Genes bound to the gene-binding carriers are not eluted in the washing process.

In step S108, genes are eluted. The gene is eluted from the gene-binding carrier using an eluting solution. Water or a low-salt concentration solution is used as an eluting solution.

In step S109, genes are multiplied and detected. Genes are multiplied by the polymerase chain reaction. A primer (single strand DNA having the same base sequence as about 20 bases at both ends of an intended DNA area), enzyme for synthesis of DNA (polymerase), four types of substrate (dNTP) and the like are added to the eluted genes and a temperature cycle is applied. Thermal denaturation, annealing, and synthesis of complementary strands are thereby caused.

Genes are detected while applying the temperature cycle. The sample is irradiated with excitation light. Genes are fluorescent-labeled in advance. By detecting fluorescence, genes can be detected.

In step S110, the analysis chip 100 is disposed of. The analysis chip 100 is taken out of the analyzer 300 before being disposed of.

An example of the analysis chip 100 according to the present invention will be described more specifically with reference to FIGS. 3 to 7. First, the example will be described with reference to FIG. 3. The analysis chip 100 in the present embodiment includes four reagent storage tanks 120, 130, 140, and 150, a sample reservoir 110, four reagent storage tanks 160, 170, 180, and 190, a gene extraction area 210, a waste liquid tank 220, a reaction tank 230, and two reagent storage tanks 240 and 250. These components are mutually connected by channels.

The channel between the four reagent storage tanks 120, 130, 140, and 150 and the sample reservoir 110 is provided with an injection side valve 113. The channel between the sample reservoir 110 and the four reagent storage tanks 160, 170, 180, and 190 is provided with a discharge side valve 114.

The four reagent storage tanks 120, 130, 140, and 150 are connected to pressure ports 121, 131, 141, and 151 respectively.

A pressure port 115 is connected to the channel between the injection side valve 113 and the sample reservoir 110. The sample reservoir 110 is provided with a sample inlet 111. An exhaust port 116 is connected to the sample reservoir 110.

The four reagent storage tanks 160, 170, 180, and 190 are connected to pressure ports 161, 171, 181, and 191 respectively. Pressure exhaust ports 221 and 231 are connected to the waste liquid tank 220 and the reaction tank 230 respectively. Pressure ports 241 and 251 are connected to the reagent storage tanks 240 and 250 respectively.

Here, the side on which the reagent storage tanks 120, 130, 140, and 150 are located is called the upstream side relative to the sample reservoir 110 and the side on which the waste liquid tank 220 and the reaction tank 230 are located is called the downstream side.

In the analysis chip of the present embodiment, a liquid is caused to move from the upstream side to the downstream side by using a gas under pressure. The pressure source of the analyzer is connected to the pressure port on the upstream side of the liquid and the low pressure source of the analyzer is connected to the exhaust port on the downstream side of the liquid. The gas under pressure from the pressure port squeezes out the liquid. After squeezing out the liquid, the gas under pressure is discharged from the exhaust port.

The gas under pressure is a dry inert gas under a predetermined pressure, for example, nitrogen, helium, argon and the like. Oxygen and carbon dioxide are not used because oxygen could oxidizes reagents and carbon dioxide could change pH of reagents. The low pressure source of the analyzer may be an atmospheric pressure.

The structure of the injection side valve 113 and discharge side valve 114 will be described. The injection side valve 113 and discharge side valve 114 have a polymer filled in the channel. The polymer dissolves at 100° C. or below and solidifies at room temperature. A polymer compatible with a biological sample is selected. Such a polymer includes agarose gel. Particularly, low melting point agarose gel, which dissolves at about 65° C. and solidifies at room temperature, is preferable.

When producing the injection side valve 113 or discharge side valve 114, the channel is filled with a dissolved polymer and cooled down to the room temperature. The polymer is thereby solidified and the channel is closed. To open the channel, the valve is locally heated to dissolve the polymer. The dissolved polymer moves along the inner wall of the channel. When the channel is opened, the polymer is cooled down to the room temperature. The polymer is thereby solidified while it adheres to the inner wall of the channel. The valve in the present embodiment performs an irreversible operation of never being closed again once it is opened.

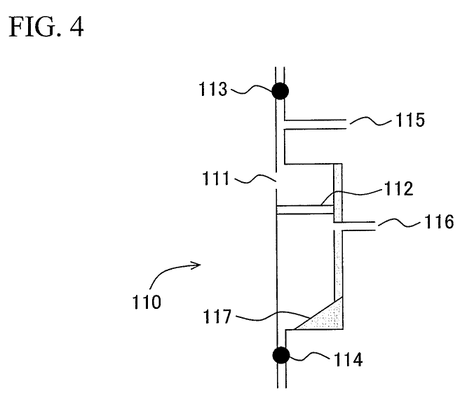

An example of the structure of the sample reservoir 110 will be described with reference to FIG. 4. A filter 112 is provided inside the sample reservoir 110. The filter 112 removes crushed substance larger than bacteria. If for example, bacteria are assumed to have a dimension of 20 μm, it is preferable that the filter 112 has an opening of 20 μm or more.

The sample inlet 111 is provided above the filter 112 and the exhaust port 116 is provided below the filter 112. A taper is formed at a bottom 117 of the sample reservoir 110. The taper prevents a sample from being left at the bottom 117 of the sample reservoir 110.

FIG. 3 is referred to again for a description that follows. The gene extraction area 210 is filled with gene-binding carriers. For example, silica wool, glass wool, glass fiber, a glass bead or the like is used as a gene-binding carrier. When the glass bead is used, it is preferable that the bead diameter is 50 μm or less to increase the contact area. If the bead diameter is too small, gene-binding carriers could flow out from the gene extraction area 210 to the channel. Thus, the bead diameter is optimally 20 to 30 μm.

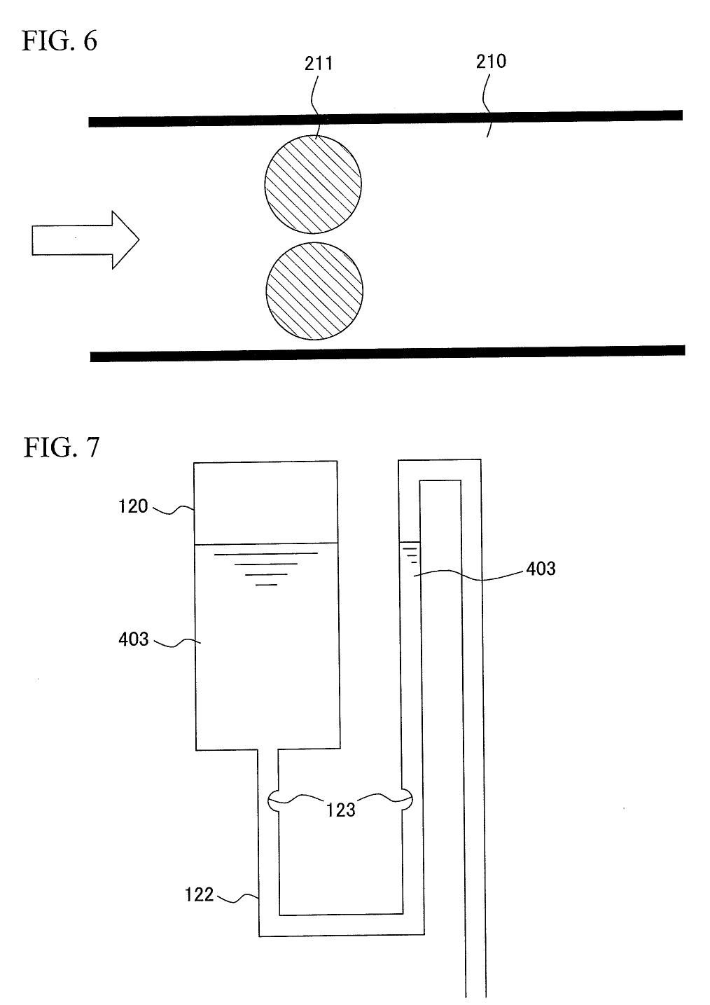

An example of the structure of the gene extraction area 210 will be described with reference to FIG. 6. The gene extraction area 210 is provided with a weir 211 so that gene-binding carriers should not flow out to the channel. The weir 211 includes a plurality of pillar shaped bodies formed in the gene extraction area 210. A channel is formed by a gap between pillar shaped bodies and a gap between a pillar shaped body and the inner wall. The width of the channel is narrower than the bead diameter. If the width of the channel is less than 10 μm, fluid resistance increases and thus, smooth flow of the sample is made difficult. Therefore, the width of the channel formed by the weir 211 is preferably 15 to 50 μm.

Next, a material of the analysis chip 100 and a method of producing it will be described. The analysis chip has a thin rectangular disk shape formed by pasting two boards together. The first board has grooves constituting channels, reagent storage tanks, the sample reservoir 110, the gene extraction area 210 and the like formed thereon, and the second board has holes constituting ports formed therein. Grooves of the first board are created by transfering resin molds created by stereolithography. Resin molds created by stereolithography are not smooth curves. Thus, molds have basically a rectangular structure. Therefore, the cross section of grooves formed on the first board is rectangular. If a reagent is flown through a channel having a rectangular cross section, the reagent may adhere to corners of the channel and remain there. A carryover in which the reagent remaining in the channel mixes with the reagent that flows through the channel next occurs. The cross section of the channel may be made smaller to suppress adhesion of the reagent to the channel wall. In the present embodiment, the cross section of the channel was set to 0.5 mm horizontally and 0.5 mm vertically to suppress the carryover of reagent.

The second board is created by forming holes in a plate material. The analysis chip 100 is produced by pasting the second board on the first board together.

A transparent material such as glass and resin is used as a material of the board. Glass is expensive in process costs and also breaks easily. Resin is superior in processing characteristics and disposability. Therefore, resin is preferably used as a material of the board. Molds of resin are not particularly limited, but it is preferable to have the following characteristics:

(1) Biocompatibility is excellent. Ordinary silicon rubber is physiologically inactive.

(2) Molds can be transferred with precision of submicron. The resin preferably has low viscosity and rich liquidity before hardening and is capable of penetrating into detailed forms of complex molds satisfactorily.

(3) The resin is inexpensive. It is preferable, for example, that the resin costs 8 yen per gram. Pyrex glass, which is conventionally a general-purpose micro-device material, costs 1k yen per gram. Therefore, it is preferable that the resin costs 1/100 or less the price of Pyrex glass.

(4) The resin is easily disposable by burning. Glass cannot be burnt and thus, is not preferable.

Materials having such characteristics include polydimethylsiloxane (PDMS: manufactured by Dow Corning Asia, Sylgard 184).

FIG. 3 is referred to again for a description that follows. A case in which microorganisms contained in a solid sample are detected using the analysis chip in the present embodiment will be described. The reagent storage tanks 120, 130, 140, and 150 contain a culture solution, a first enzyme acting as a cell wall dissolution solution, a second enzyme acting as a cell wall dissolution solution, and a chaotropic ion solution respectively. Therefore, these reagent tanks will be called hereafter the culture solution storage tank 120, first enzyme storage tank 130, second enzyme storage tank 140, and chaotropic storage tank 150 respectively. The reagent storage tanks 160, 170, 180, and 190 contain a first washing solution, a second washing solution, a third washing solution, and a gene eluting solution respectively. Therefore, these reagent tanks will be called hereafter the first washing solution storage tank 160, second washing solution storage tank 170, third washing solution storage tank 180, and eluting solution storage tank 190 respectively. The reagent storage tanks 240 and 250 contain a first gene amplification reagent and a second gene amplification reagent respectively. Therefore, these reagent tanks will be called hereafter the first gene amplification reagent storage tank 240 and second gene amplification reagent storage tank 250 respectively.

In the present embodiment, it is preferable that the volume of the culture solution storage tank 120 is 20 to 100 μL, that of the first enzyme storage tank 130 is 20 to 100 μL, that of the second enzyme storage tank 140 is 5 to 20 μL, and that of the chaotropic storage tank 150 is 400 to 800 μL. It is also preferable that the volume of the first washing solution storage tank 160 is 200 to 300 μL, that of the second washing solution storage tank 170 is 50 to 150 μL, that of the third washing solution storage tank 180 is 50 to 100 μL, that of the eluting solution storage tank 190 is 10 to 20 μL, that of the first gene amplification reagent storage tank 240 is 20 to 40 μL, and that of the second gene amplification reagent storage tank 250 is 10 to 20 μL.

These reagent storage tanks are formed like a slender channel shape. To feed a reagent in a reagent storage tank, a gas under pressure is fed into the reagent storage tank from behind the reagent storage tank. If the reagent storage tank does not have a slender channel shape, the reagent is squeezed out only from regions where the gas easily passes through and the reagent in other portions remains in the reagent storage tank. It is reasonable to adopt a reagent storage tank in a channel shape to effectively reduce the amount of reagent to be consumed.

The cross sectional shape of these reagent storage tanks preferably has a ratio of height to width being equal to or less than 10. If the ratio of height to width exceeds 10, the resin in a ceiling portion of the channel may be bent, leading to disintegration of the rectangular structure of the channel. The cross section of the channel may thereby be narrowed, making it difficult to feed a reagent. The cross sectional shape of the reagent storage tank in the present embodiment is set to 1 mm horizontally and 2 to 3 mm vertically. The cross section of a channel connected to a reagent storage tank is smaller than that of the reagent storage tank. For example, the cross section of such a channel is preferably ¼ or less of that of the reagent storage tank.

Mixing a sample or the first washing solution into the eluting solution makes detection of genes difficult. Thus, the eluting solution storage tank 190 is preferably arranged apart from both the sample reservoir 110 and first washing solution storage tank 160.

In the present embodiment, the analysis chip 100 is provided to a user in a cold state or a frozen state. By preserving the analysis chip 100 in a cold state at 4° C., activity of the reagent can be maintained for one month. By preserving the analysis chip 100 in a frozen state at −20° C., activity of the reagent can be maintained for half a year or longer.

The analysis chip 100 contains only reagents for one inspection. The analysis chip 100 is disposable after one inspection. Therefore, there is no wasteful use of reagents, improving economical efficiency. In addition, there is no need for the user to dispense reagents into each reagent storage tank, not only reducing time, but also preventing contamination.

In the present embodiment, each of the analysis chips 100 contains 10 kinds of reagents for one inspection. That is, the culture solution, first and second cell wall dissolution solutions, chaotropic ions, first, second, and third washing solutions, gene eluting solution, and first and second gene amplification reagents are contained in the culture solution storage tank 120, first enzyme storage tank 130, second enzyme storage tank 140, chaotropic storage tank 150, first washing solution storage tank 160, second washing solution storage tank 170, third washing solution storage tank 180, eluting solution storage tank 190, first gene amplification reagent storage tank 240, and second gene amplification reagent storage tank 250 respectively.

As the culture solution, bouillon including alanine, adenosine, glucose and the like is preferable. Particularly, bouillon containing 1 to 10 mM of L-alanine is optimal. As the first cell wall dissolution solution, that is the first enzyme, lysozyme or achromopeptidase is suitable. As the second cell wall dissolution solution, that is the second enzyme, protease K is suitable.

Chaotropic ions include guanidine thiocyanate, guanidine hydrochloric acid, sodium iodide, potassium bromide and the like. Guanidine hydrochloric acid is suitable because composition change due to cool storage or frozen storage is extremely small. In addition, it is preferable to make chaotropic ions contain a surfactant or buffer. The surfactant is not particularly limited, but includes Tween-20, Triton X-100 and the like. The buffer is not particularly limited, but includes tris-hydrochloride, potassium dihydrogen phosphate-sodium tetraborate and the like. By adding chaotropic ions, the cell membrane of bacteria is more thoroughly destroyed. In addition, chaotropic ions denature many proteins contained in a sample to inhibit action of nuclease (enzyme to decompose nucleic acid).

As the first washing solution, chaotropic ions such as guanidine thiocyanate, guanidine hydrogen chloride, sodium iodide, and potassium bromide are preferable. As the second washing solution, an ethanol or potassium acetate solution of high concentration of 50% or more is preferable. As the third washing solution, a solution obtained by diluting the second washing solution twofold or more (diluted solution of the ethanol or potassium acetate solution), or sterilized distilled water is preferable.

As the eluting solution, sterilized distilled water or a buffer solution such as TRIS-EDTA and TRIS-acetate can be used. The first gene amplification reagent includes four dNTP (dATP, dCTP, dGTP, and dTTP), a buffer (such as TRIS hydrochloric acid, KCl, and MgCl₂), a primer and the like. The second gene amplification reagent includes an enzyme for synthesis of DNA (such as Taq DNA polymerase, Tth DNA polymerase, Vent DNA polymerase, and thermosequenase), fluorochrome (such as ethidium bromide, SYBR GREEN (manufactured by Molecular Probe), FAM, and ROX) and the like.

Next, processing for detecting presence/absence of bacteria to be analyzed will be described. The user first brings the analysis chip 100 in cool storage back to the room temperature or unfreezes the analysis chip 100 in frozen storage at room temperature.

Sample crushing and the generation processing of a suspension in step S101 are performed. As described above, the sample is cut into pieces of appropriate size before being put into a crush bag. Water is added to the bag to crush the sample. A suspension of the sample is thereby generated.

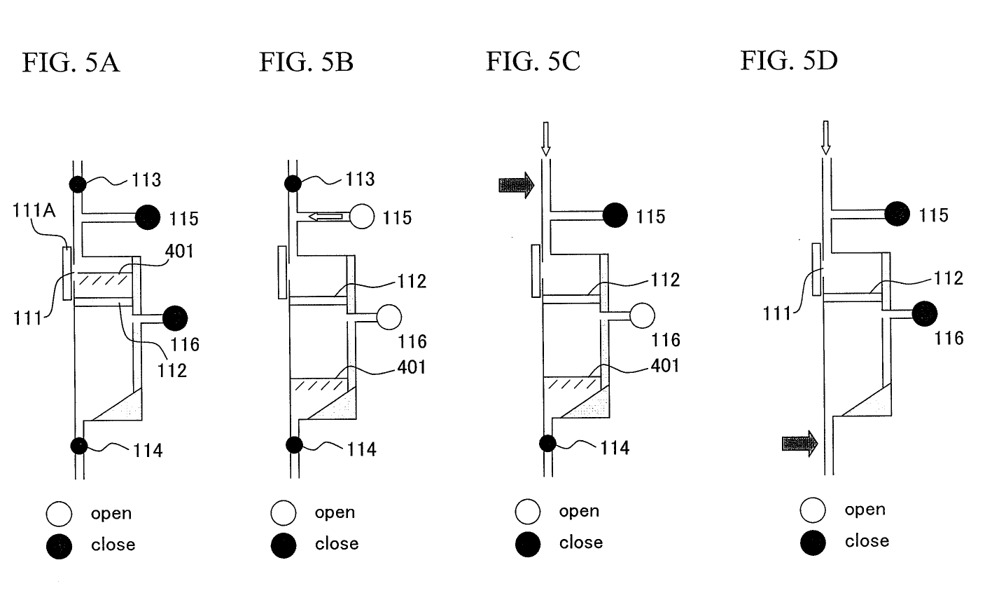

Extraction of the sample in step S102 is performed. As shown in FIG. 5A, a portion of the suspension is injected into the sample reservoir 110 via the sample inlet 111. The amount of a sample 401 is about 100 μL. The sample inlet 111 is closed by a cover 111A to seal the sample reservoir 110. The cover 111A is preferably a thin sheet made of the same material as that of the analysis chip 100. Since the board and sheet are formed of the same resin, both adhere closely to each other and the sample reservoir 110 can be sealed easily. Though the processing to cover the sample inlet 111 may be performed manually, it is preferable that the analyzer 300 is provided with a mechanism to attach a cover to the sample inlet 111. Incidentally, at this point, all ports and the valves 113 and 114 are closed.

The removal process of crushed substance in step S103 is performed. As shown in FIG. 5A, the suspension of the sample 401 is injected on the upper side of the filter 112 inside the sample reservoir 110. As shown in FIG. 5B, the exhaust port 116 connected to the sample reservoir 110 is connected to the low pressure source and the pressure port 115 on the upstream side of the sample reservoir 110 is connected to the pressure source. All ports except the two ports 115 and 116 are closed. A gas under pressure is introduced to the sample reservoir 110 from the pressure port 115. The sample 401 is thereby passed through the filter 112 before being accumulated at the bottom of the sample reservoir 110. Crushed substance larger than a dimension of bacteria is removed by the filter 112. If for example, bacteria are assumed to have a dimension of 20 μm, substance whose dimension is 20 μm or larger will be removed.

Next, the cultivation process of bacteria in step S104 is performed. The pressure port 115 in the sample reservoir 110 is closed. The injection side valve 113 is locally heated to dissolve filling material inside the injection side valve 113. As shown in FIG. 5C, the injection side valve 113 is thereby opened.

The pressure port 121 connected to the culture solution storage tank 120 is connected to the pressure source and the exhaust port 116 connected to the sample reservoir 110 is connected to the low pressure source. All ports except the two ports 121 and 116 are closed.

A gas under pressure from the pressure port 121 squeezes out 100 μL of culture solution contained in the culture solution storage tank 120. The culture solution squeezed out by the gas under pressure reaches the sample reservoir 110. The gas under pressure is discharged to the low pressure source of the exhaust port 116. The culture solution passes through the filter 112 before reaching the bottom of the sample reservoir 110. There, the culture solution mixes with the sample.

Bacteria in the sample are cultivated by the culture solution. As described above, the optimal culture solution is bouillon containing 1 to 10 mM of L-alanine. Cultivation of 30 minutes or longer is preferable and, depending on target bacteria, cultivation of 3 to 4 hours is preferable. The cultivation temperature of 35 to 40° C. is preferable and particularly preferable is 35 to 37° C.

Next, the dissolution process of bacteria in step S105 is performed. The pressure port 121 of the culture solution storage tank 120 is closed and the pressure port 131 connected to the first enzyme storage tank 130 is connected to the pressure source. The exhaust port 116 of the sample reservoir 110 is connected to the low pressure source. All ports except the two ports 131 and 116 are closed.

A gas under pressure from the pressure port 131 squeezes out 100 μL of first enzyme contained in the first enzyme storage tank 130. The first enzyme squeezed out by the gas under pressure reaches the sample reservoir 110. The gas under pressure is discharged to the low pressure source of the exhaust port 116. The first enzyme passes through the filter 112 before reaching the bottom of the sample reservoir 110. There, the first enzyme mixes with the sample.

Enzyme treatment by the first enzyme is performed. As described above, the first enzyme is preferably lysozyme or achromopeptidase. The cell wall, which is an outer part of bacteria, is dissolved by the first enzyme. Enzyme treatment preferably lasts 10 minutes or longer. The optimum temperature of enzyme treatment is 37° C.

Next, the pressure port 131 of the first enzyme storage tank 130 is closed and the pressure port 141 connected to the second enzyme storage tank 140 is connected to the pressure source. The exhaust port 116 of the sample reservoir 110 is connected to the low pressure source. All ports except the two ports 141 and 116 are closed.

A gas under pressure from the pressure port 141 squeezes out 20 μL of second enzyme contained in the second enzyme storage tank 140. The second enzyme squeezed out by the gas under pressure reaches the sample reservoir 110. The gas under pressure is discharged to the low pressure source of the exhaust port 116. The second enzyme passes through the filter 112 before reaching the bottom of the sample reservoir 110. There, the second enzyme mixes with the sample.

Enzyme treatment by the second enzyme is performed. As described above, the second enzyme is preferably protease K. The cell membrane, which is inside the cell wall of bacteria, is dissolved by the second enzyme and genes of the bacteria are released out of the bacteria. Enzyme treatment preferably lasts 10 minutes or longer. The optimum temperature of enzyme treatment is 37 to 60° C.

Next, the pressure port 141 of the second enzyme storage tank 140 is closed and the pressure port 151 connected to the chaotropic storage tank 150 is connected to the pressure source. The exhaust port 116 of the sample reservoir 110 is connected to the low pressure source. All ports except the two ports 151 and 116 are closed.

A gas under pressure from the pressure port 151 squeezes out 800 μL of chaotropic ion solution contained in the chaotropic storage tank 150. The chaotropic ion solution squeezed out by the gas under pressure reaches the sample reservoir 110. The gas under pressure is discharged to the low pressure source of the exhaust port 116. The chaotropic ion solution passes through the filter 112 before reaching the bottom of the sample reservoir 110. There, the chaotropic ion solution mixes with the sample.

As described above, the chaotropic ion solution is preferably guanidine hydrochloric acid. By adding chaotropic ions, the cell membrane of bacteria is more thoroughly destroyed.

Next, the capture process of bacteria in step S106 is performed. The pressure exhaust port 221 connected to the waste liquid tank 220 is connected to the low pressure source. A back pressure is applied to the pressure exhaust port 231 connected to the reaction tank 230. The back pressure is lower than the pressure of the high pressure source and higher than that of the lower pressure source. The discharge side valve 114 is locally heated to dissolve filling material inside the discharge side valve 114. As shown in FIG. 5D, the discharge side valve 114 is thereby opened. The exhaust port 116 of the sample reservoir 110 is closed. The pressure port 151 of the chaotropic storage tank 150 is connected to the high pressure source. All ports except the three ports 221, 231, and 151 are closed.

A gas under pressure from the pressure port 151 reaches the bottom of the sample reservoir 110 via the filter 112 of the sample reservoir 110. The gas under pressure squeezes out the sample accumulated at the bottom of the sample reservoir 110. The gas under pressure is discharged to the low pressure source of the pressure exhaust port 221. The sample squeezed out by the gas under pressure reaches the gene extraction area 210.

Genes of bacteria in the sample are bound to gene-binding carriers filled in the gene extraction area 210 by action of the chaotropic ion on the sample. To facilitate binding of bacteria genes and gene-binding carriers, the time taken for the sample to pass through the gene extraction area 210 is preferably 10 minutes or more.

To cause the sample to move through fine channels of the gene-binding carriers in such a manner that it takes 10 minutes or more for the sample to pass through there, advanced feeding control is needed. Therefore, the sample may be fed intermittently.

After passing through the gene extraction area 210, the sample accumulates in the waste liquid tank 220. Since a back pressure is applied to the pressure exhaust port 231 of the reaction tank 230, the sample will not enter the reaction tank 230.

Next, the washing process of genes in step S107 is performed. First, washing by the first washing solution is performed. The pressure port 151 of the chaotropic storage tank 150 is closed and the pressure port 161 connected to the first washing solution storage tank 160 is connected to the pressure source. The pressure exhaust port 221 of the waste liquid tank 220 is connected to the low pressure source and a back pressure is applied to the pressure exhaust port 231 of the reaction tank 230. All ports except the three ports 161, 221, and 231 are closed.

A gas under pressure from the pressure port 161 squeezes out 200 μL of first washing solution contained in the first washing solution storage tank 160. The gas under pressure is discharged to the low pressure source of the pressure exhaust port 221. The first washing solution squeezed out by the gas under pressure passes through the gene extraction area 210. Proteins remaining in the gene extraction area 210 are thereby removed. The first washing solution is stored in the waste liquid tank 220 after washing. Since a back pressure is applied to the pressure exhaust port 231 of the reaction tank 230, the first washing solution will not enter the reaction tank 230.

Next, washing by the second washing solution is performed. The pressure port 161 of the first washing solution storage tank 160 is closed and the pressure port 171 connected to the second washing solution storage tank 170 is connected to the pressure source. The pressure exhaust port 221 of the waste liquid tank 220 is connected to the low pressure source and a back pressure is applied to the pressure exhaust port 231 of the reaction tank 230. All ports except the three ports 171, 221, and 231 are closed.

A gas under pressure from the pressure port 171 squeezes out 150 μL of second washing solution contained in the second washing solution storage tank 170. The gas under pressure is discharged to the low pressure source of the pressure exhaust port 221. The second washing solution squeezed out by the gas under pressure passes through the gene extraction area 210. Chaotropic ions remaining in the gene extraction area 210 are thereby removed. The second washing solution is stored in the waste liquid tank 220 after washing. Since a back pressure is applied to the pressure exhaust port 231 of the reaction tank 230, the second washing solution will not enter the reaction tank 230.

Lastly, washing by the third washing solution is performed. The pressure port 171 of the second washing solution storage tank 170 is closed and the pressure port 181 connected to the third washing solution storage tank 180 is connected to the pressure source. The pressure exhaust port 221 of the waste liquid tank 220 is connected to the low pressure source and a back pressure is applied to the pressure exhaust port 231 of the reaction tank 230. All ports except the three ports 181, 221, and 231 are closed.

A gas under pressure from the pressure port 181 squeezes out 100 μL of third washing solution contained in the third washing solution storage tank 180. The gas under pressure is discharged to the low pressure source of the pressure exhaust port 221. The third washing solution squeezed out by the gas under pressure passes through the gene extraction area 210. The second washing solution remaining in the gene extraction area 210 is thereby removed. The third washing solution is stored in the waste liquid tank 220 after washing. Since a back pressure is applied to the pressure exhaust port 231 of the reaction tank 230, the third washing solution will not enter the reaction tank 230.

Next, the elution process of genes in step S108 is performed. The pressure port 181 of the third washing solution storage tank 180 is closed and the pressure port 191 connected to the eluting solution storage tank 190 is connected to the pressure source. The pressure exhaust port 231 of the reaction tank 230 is connected to the low pressure source and a back pressure is applied to the pressure exhaust port 221 of the waste liquid tank 220. The back pressure is lower than the pressure of the high pressure source and higher than that of the lower pressure source. All ports except the three ports 191, 221, and 231 are closed.

A gas under pressure from the pressure port 191 squeezes out an eluting solution contained in the eluting solution storage tank 190. The gas under pressure is discharged to the low pressure source of the pressure exhaust port 231. The eluting solution squeezed out by the gas under pressure passes through the gene extraction area 210. Genes bound to gene-binding carriers are thereby eluted. Genes are stored in the reaction tank 230 together with the eluting solution. Since a back pressure is applied to the pressure exhaust port 221 of the waste liquid tank 220, the eluting solution will not enter the waste liquid tank 220.

The amplification and detection process of genes in step S 109 is performed. First, the first gene amplification reagent is added to the sample. The pressure port 191 of the eluting solution storage tank 190 and the pressure exhaust port 221 of the waste liquid tank 220 are closed. The pressure exhaust port 231 of the reaction tank 230 is connected to the low pressure source. The pressure source is connected to the pressure port 241 connected to the first gene amplification reagent storage tank 240. All ports except the two ports 231 and 241 are closed.

A gas under pressure from the pressure port 241 squeezes out 40 μL of first gene amplification reagent contained in the first gene amplification reagent storage tank 240. The gas under pressure is discharged to the low pressure source of the pressure exhaust port 231. The first gene amplification reagent squeezed out by the gas under pressure moves to the reaction tank 230. There, the first gene amplification reagent mixes with the gene.

Next, the second gene amplification reagent is added to the sample. The pressure port 241 of the first gene amplification reagent storage tank 240 is closed and the pressure source is connected to the pressure port 251 of the second gene amplification reagent storage tank 250. The low pressure source is connected to the pressure exhaust port 231 of the reaction tank 230. All ports except the two ports 231 and 251 are closed.

A gas under pressure from the pressure port 251 squeezes out 20 μL of second gene amplification reagent contained in the second gene amplification reagent storage tank 250. The gas under pressure is discharged to the low pressure source of the pressure exhaust port 231. The second gene amplification reagent squeezed out by the gas under pressure moves to the reaction tank 230. There, the second gene amplification reagent mixes with the gene.

A temperature cycle that periodically changes the temperature of the reaction tank 230 is applied in order to amplify genes and detect them inside the reaction tank 230.

As an example of the temperature cycle, a method of alternately repeating “90 to 95° C. for 10 to 30 seconds” and “65 to 70° C. for 10 to 30 seconds” 30 to 45 times is known. As a preferred example of the temperature cycle, a method of alternately repeating “94° C. for 30 seconds” and “68° C. for 30 seconds” 45 times is known.

The reaction tank 230 is irradiated with excitation light while executing the temperature cycle. If a gene has a fluorochrome intercalated inside double strands, absorbed light energy is transferred to the fluorochrome. As a result, the fluorochrome is excited to emit fluorescence. When objective genes in the sample are amplified, the amount of fluorescence increases. Therefore, presence/absence of the objective genes can be known in real time during temperature cycle by detecting the amount of fluorescence from the reaction tank 230.

Incidentally, the analysis chip 100 is held while being kept standing in the analyzer 300. Therefore, if a portion of reactant evaporates during temperature cycle, vapor stays in an upper part of the reaction tank 230. Thus, the reaction tank 230 will not have its sides fogged by vapor. Consequently, detection sensitivity of genes will not decrease. This is an advantage of the vertical type.

When detection of genes is finished, disposal of the analysis chip in step S110 is carried out.

FIG. 7 is referred to for a description that follows. As described above, the analysis chip is held while being kept standing in the analyzer in the present invention. In this case, a channel 122 connected to the reagent storage tank 120 is formed in a U shape so that a reagent 403 inside the reagent storage tank 120 should not flow out due to its own weight. Preferably, a weir 123 is provided in the channel 122. Reagents stored in each reagent storage tank can thereby be prevented from flowing out more reliably.

The analyzer 300 will be described with reference to FIGS. 8 and 9. FIG. 8 shows an appearance of the analyzer 300 and FIG. 9 shows an internal structure of the analyzer 300. The analyzer 300 includes an analysis chip insertion system, a temperature control system, a pressure gas supply system, and an optical detection system.

The analysis chip insertion system will be described. The analysis chip insertion system has a front cover 301, a board 310, a chip holder 320, and a chip stopper 322. To insert the analysis chip 100 into the analyzer 300, the front cover 301 is opened and the analysis chip 100 is inserted along a chip guide provided on the front cover 301. When a lower end of the analysis chip 100 comes into contact with the chip stopper 322, the front cover 301 is closed. The analysis chip 100 is held by being kept standing and being sandwiched between the board 310 and the chip holder 320.

A plurality of tubes 312 are formed on the board 310. These tubes 312 are connected to the pressure ports, exhaust port, and pressure exhaust ports of the analysis chip 100.

The temperature control system will be described. The temperature control system has a temperature control part 311 provided on the board 310 and a heater 321 provided on the chip holder 320. The temperature control part 311 maintains the analysis chip 100 at a predetermined temperature. That is, the temperature control part 311 functions to maintain the sample reservoir and reaction tank at a predetermined temperature. The heater 321 is used to open the injection side valve 113 and discharge side valve 114. The valve is locally heated by the heater 321 to dissolve filling material.

The structure of the temperature control part 311 and that of the heater 321 are not particularly limited, but a Peltier device that can readily raise and lower the temperature by simply changing the direction of applied current is suitable.

The pressure gas supply system will be described. The pressure gas supply system has a switching valve 330, a valve control part 331, a pressure pump 340, and a pump control part 341. The tube 312 of the board 310 and each port of the switching valve are connected by a tube 302. It is sufficient for the pressure pump 340 to have a function to supply a gas under pressure to squeeze out a liquid and there is no need to provide a high-precision flow rate. The pressure pump 340 only supplies a gas under pressure or a back pressure and does not suck in a gas. Therefore, the pressure pump 340 may be simple and compact.

The pump 340 operates in high pressure operation mode to supply a high pressure source or in back pressure mode to supply a back pressure depending on a control signal from the pump control part 341. Each port of the switching valve 330 is closed, opened to the air, or connected to the pump depending on a control signal from the valve control part 331.

To close a port of the analysis chip, it is sufficient to close a port of the switching valve 330 connected to the port. To connect a port of the analysis chip to the low pressure source, it is sufficient to open a port of the switching valve 330 connected to the port, to the air. To connect a port of the analysis chip to the high pressure source, it is sufficient to connect a port of the switching valve 330 connected to the port, to a pump being operated in high pressure operation mode. To connect a port of the analysis chip to a back pressure, it is sufficient to connect a port of the switching valve 330 connected to the port, to a pump being operated in back pressure operation mode.

The optical detection system has a light source 350, a light source control part 351, an excitation filter 355, a mirror 360, a filter 375, a photodetector 370, a photodetector control part 371, and a light signal converter 380. The light source 350 is preferably a xenon lamp with a wide wavelength region, but may be a light emitting diode (LED) when a specific wavelength is needed. The photodetector 370 may be a CCD camera, a photomultiplier, a photodiode or the like, but is preferably a photodiode which is compact.

An excitation light of a specific wavelength region is generated by having excitation light from the light source 350 pass through the excitation filter 355. Genes inside the reaction tank of the analysis chip are irradiated with the excitation light. Genes are fluorescent-labeled in advance. Fluorescence from genes is reflected by the mirror 360 to change its optical path before being incident on the filter 375. Fluorescence whose unnecessary wavelength components have been removed by the filter 375 is detected by the photodetector 370. A detection signal from the photodetector 370 is converted into a digital signal by the light signal converter 380. The amount of microorganisms inside the reaction tank of the analysis chip is displayed in this manner on a display 390.

The analyzer 300 further has the display 390 provided on the front side, a power unit 305 provided on the rear side, and a controller (not shown). Signals are supplied from the controller to a temperature control part 315, the valve control part 331, the pump control part 341, the light source control part 351, and the photodetector control part 371.

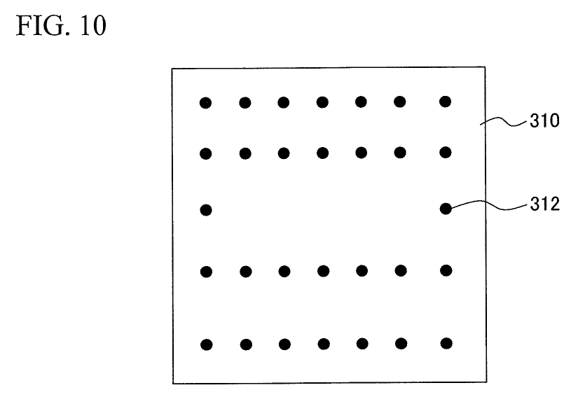

FIG. 10 exemplifies the board 310 of the analyzer. The board 310 is provided with a plurality of channels. These channels are provided corresponding to port positions of the analysis chip. By providing the many tubes 312 on the board 310 in advance, the analysis chip 100 can be inserted into the analyzer even if the format of the analysis chip 100 changes. That is, the analyzer 300 can use the various analysis chips 100 with different port positions. Accordingly, the analyzer 300 could become a platform for the various analysis chips 100.

One reaction tank 230 is provided in the analysis chip 100 shown in FIG. 3. However, a plurality of the reaction tanks 230 may be provided when a plurality of bacteria should be detected simultaneously. In this case, a primer corresponding to bacteria to be detected is needed. Thus, a plurality of the first gene amplification reagent storage tanks and a plurality of the second gene amplification reagent storage tanks are also needed. Further, an optical system that sequentially irradiates the plurality of the reaction tanks with excitation light is needed. However, there is an advantage that a plurality of bacteria can simultaneously be detected on one analysis chip.

In the microorganism detection system in the present embodiment, the switching valve 330 of the pressure gas supply system is provided in the analyzer 300, instead of inside the analysis chip 100. Accordingly, there is no mechanical component contained in the analysis chip 100. Thus, miniaturization, lower prices, and disposability can be achieved.

A compact and portable analyzer can be provided by simply combining a board holding an analysis chip containing no mechanical component and a photodetector.

In addition, the reagent storage tanks, reaction tank, and channels can be made finer by fine processing. The amount of reagents and samples can thereby be made smaller, leading to lower costs. Further, there are advantages such as faster temperature control, faster mixing, and uniform reaction.

In the microorganism detection system in the present embodiment, the analysis chip 100 and the analyzer 300 are combined for use. Thus, sample filtering, bacteria cultivation, bacteria dissolution, gene extraction, and multiplication and detection of genes can be performed automatically inside a compact analysis chip. After injecting a sample into the analysis chip, all processing can be performed automatically by the analyzer. Therefore, anybody can detect bacteria safely.

Further, the chip is provided to the user in a cold state or a frozen state by storing reagents for one inspection in a disposable chip in advance. Genes and bacteria can thereby be detected extremely easily and swiftly. Further, after analyzing genes, the samples can be disposed of as waste together with reagents.

Next, a second embodiment of the analysis chip of the present embodiment will be described. The analysis chip in the present embodiment has basically the same structure as that of the first embodiment described with reference to FIG. 3, and here only differences will be described. In the present embodiment, a piezoelectric element such as a crystal resonator and a surface acoustic wave device is arranged at the bottom of the reaction tank 230 of the analysis chip 100. The oscillating frequency of the piezoelectric element changes, depending on the weight of substance attached on its electrodes. Therefore, by detecting any change in the oscillating frequency of the piezoelectric element, the weight of substance attached on the electrodes can be detected. The piezoelectric element can continuously detect changes in a very small amount of weight in a reaction atmosphere.

The piezoelectric element arranged at the bottom of the reaction tank 230 fixes nucleotides having a known base sequence. The method of fixing may be as follows: First, a thin glass film is formed on the electrodes of the piezoelectric element by a method of sputtering, vapor deposition or the like. The glass preferably has SiO₂, which has an excellent adhesive property to electrode materials such as chrome and titanium, as a principal component. Aminopropyl trimethoxysilane (APS) is added to the thin glass film before baking at 120 to 160° C. to fix the amino group on the surface of the thin glass film. Here, it is preferable that the thickness of the electrode and that of the thin glass film are each 0.1 to 1 μm. This is because, if the thickness of one of both exceeds 1 μm, frequency response of the piezoelectric element slows down. Further, nucleotides are applied to the thin glass film coated with the amino group before the thin glass film being maintained at 37° C. and humidity 90% for one hour in a constant temperature and humidity bath. Thereafter, nucleotides are firmly fixed to the piezoelectric element by irradiating the piezoelectric element with ultraviolet radiation of 60 mJ/cm² using a UV cross linker.

Sample crushing and the generation processing of a suspension in step S101 to the elution process of genes in step S108 are the same as those in the first embodiment. The multiplication and detection process of genes in step S109 is different from that in the first embodiment.

The multiplication and detection process of genes in step S109 will be described briefly. When genes are transferred to the reaction tank 230, the temperature of the reaction tank 230 is raised to approximately 94° C. by a temperature control mechanism 313. Genes are thereby thermally denatured to produce single strands. The single strands and the nucleotides fixed at the bottom of the reaction tank are bound. The oscillating frequency of the piezoelectric element is thereby changed. By measuring frequency changes, the sequence of genes complementary to the nucleotides can be read.

Frequency characteristics of the piezoelectric element change, depending on the temperature. When the piezoelectric element is used in the reaction tank 230, the frequency changes by 15 to 30 Hz when the liquid temperature changes by 1° C. Therefore, accurate control of the liquid temperature inside the reaction tank 230 is required. However, in the present embodiment, no gene amplification reagent is needed and there is no need to apply the temperature cycle. Thus, there is an advantage that the detection time will be shorter.

Embodiments of the present invention have been described, but the present invention is not limited to the above embodiments and a person skilled in the art will readily understand that various modifications can be made to the present invention within the scope as defined by the appended claims.

What is claimed is:

1. A bacteria detection system having an analysis chip and an analyzer, wherein
said analyzer comprising: an analysis chip insertion system for holding said analysis chip; a temperature control system for maintaining said analysis chip at a predetermined temperature; a pressure gas supply system having a pressure source for supplying a pressure gas to said analysis chip and a low pressure source for discharging a gas from said analysis chip; and an optical detection system for optically detecting genes generated in said analysis chip and
said analysis chip, comprising:
a sample reservoir for holding a sample; a gene extraction area connected to said sample reservoir by a channel to hold gene-binding carriers for capturing genes in the sample; a reaction tank connected to said gene extraction area by a channel to multiply the genes; a waste liquid tank connected to said reaction tank by a channel to store a waste liquid; reagent storage tanks connected to said sample reservoir, gene extraction area, and reaction tank by channels to hold reagents; pressure ports connected to the pressure source; and an exhaust port connected to said low pressure source, wherein said analysis chip is constituted in such a way that a liquid is caused to transfer from an upstream side to a downstream side by discharging a gas under pressure from said pressure ports to said exhaust port, and
a filter to remove crushed substance of a predetermined size from the sample injected into said sample reservoir is provided in said sample reservoir.

2. The bacteria detection system according to claim 1, wherein said filter removes substance of 20 μm or larger.

3. The bacteria detection system according to claim 1, wherein
a valve is each provided in a channel connected to the upstream side of said sample reservoir and in a channel connected to the downstream side of said sample reservoir, said valve having said channel and a polymer filled in said channel, and said polymer solidifies at room temperature and is dissolved at a temperature higher than room temperature and of 100° C. or lower.

4. The bacteria detection system according to claim 1, wherein
said analysis chip insertion system of said analyzer has a board on which a plurality of channels connected to the pressure ports and exhaust port of said analysis chip are formed and a chip holder for pressing the analysis chip arranged on said board.

5. The bacteria detection system according to claim 1, wherein
said analysis chip is held while being kept standing in said analyzer.

6. An analysis chip having a sample reservoir for holding a sample, a gene extraction area connected to said sample reservoir by a channel to hold gene-binding carriers for capturing genes in the sample, a reaction tank connected to said gene extraction area by a channel to multiply the genes, a waste liquid tank connected to said reaction tank by a channel to store a waste liquid, reagent storage tanks connected to said sample reservoir, gene extraction area, and reaction tank by channels to hold reagents, pressure ports connected to a pressure source, and an exhaust port connected to a low pressure source, and being constituted in such a way that a liquid is caused to transfer from an upstream side to a downstream side by discharging a gas under pressure from said pressure ports to said exhaust port, wherein
a filter to remove crushed substance of a predetermined size from the sample injected into the sample reservoir is provided in the sample reservoir.

7. The analysis chip according to claim 6, wherein
valves are provided in a channel connected to the upstream side of said sample reservoir and in a channel connected to the downstream side of said sample reservoir, said valves having the channel and a polymer filled in said channel, and said polymer solidifies at room temperature and is dissolved at a temperature higher than room temperature and of 100° C. or lower.

8. The analysis chip according to claim 6, wherein said liquid transfer mechanism has the pressure ports connected to the pressure source and the exhaust port connected to the low pressure source, and is constituted in such a way that the liquid is caused to transfer from the upstream side to the downstream side by discharging the gas under pressure from said pressure ports to said exhaust port.

9. The analysis chip according to claim 6, wherein a piezoelectric element is provided at a bottom of said reaction tank and an amount of genes produced in said reaction tank is measured by detecting frequency changes of said piezoelectric element.

10. A bacteria detection method using an analysis chip having a sample reservoir for holding a sample, a gene extraction area connected to said sample reservoir by a channel to hold gene-binding carriers for capturing genes in the sample, a reaction tank connected to said gene extraction area by a channel to multiply said genes, a waste liquid tank connected to said reaction tank by a channel to store a waste liquid, reagent storage tanks connected to said sample reservoir, gene extraction area, and reaction tank by channels to hold reagents and an analyzer holding said analysis chip; comprising:
generating a suspension of bacteria contained in said sample in a bag by putting the sample and distilled water into said bag and hitting said bag;
extracting a predetermined amount of sample from said bag, injecting the extracted sample into said sample reservoir of said analysis chip, and sealing said sample reservoir;
causing said sample to pass through a filter of said sample reservoir for filtering by connecting a high pressure source to an upstream side of said sample reservoir and a low pressure source to a downstream side of said sample reservoir;
introducing a culture solution stored in said reagent storage tank into said sample reservoir by connecting the high pressure source to the upstream side of said reagent storage tank in which said culture solution is stored and the low pressure source to the downstream side of said sample reservoir;
introducing a solution containing chaotropic ions stored in said reagent storage tank into said sample reservoir by connecting the high pressure source to the upstream side of said reagent storage tank in which the solution containing the chaotropic ions is stored and the low pressure source to the downstream side of said sample reservoir;
guiding the sample containing bacteria whose cell membrane has been destroyed in said sample reservoir to said gene extraction area and causing genes to bind to gene-binding carriers by connecting the high pressure source to the upstream side of said sample reservoir and the low pressure source to the downstream side of said gene extraction area;
introducing a washing solution into said gene extraction area and introducing a waste liquid of the washing solution into said waste liquid tank by connecting the high pressure source to the upstream side of said reagent storage tank in which the washing solution is stored and the low pressure source to said waste liquid tank on the downstream side of said gene extraction area;
eluting genes bound to said gene-binding carriers in said gene extraction area and introducing a solution containing said eluted genes by connecting the high pressure source to the upstream side of said reagent storage tank in which an eluting solution is stored and the low pressure source to the reaction tank on the downstream side of said gene extraction area;
introducing a gene amplification reagent stored in the reagent storage tank into said reaction tank by connecting the high pressure source to the upstream side of said reagent storage tank in which the gene amplification reagent is stored and the low pressure source to said reaction tank; and
detecting genes after the genes being multiplied in said reaction tank.
